(19) United States
(12) Patent Application Publication
(10) Pub. No.: US 2004/0127456 A1
(43) Pub. Date: Jul. 1, 2004

(54) Prevention and treatment of endotoxemia and related complications associated with surgery

(76) Inventor: Melvyn Lynn, East Brunswick, NJ (US)

Correspondence Address: CLARK & ELBING LLP, 101 FEDERAL STREET, BOSTON, MA, 02110, US

(21) Appl. No.: 10/169,628

(22) PCT Filed: Jan. 12, 2001
(86) PCT No.: PCT/US01/01273

(51) Int. Cl. A61K 31/739

(52) U.S. Cl. 514/54

(57) ABSTRACT

This invention provides methods of preventing and treating endotoxemia and related complications associated with surgical procedures, such as cardiac surgical procedures, by administration of an antiendotoxin compound.

### BACKGROUND OF THE INVENTION

This invention relates to methods of preventing and treating endotoxemia and related complications that are associated with surgical procedures.

Endotoxin, or lipopolysaccharide, is a component of the outer cell membrane of gram-negative bacteria, is shed from the membranes of growing and dying bacteria, and induces an “innate” immune response. In most cases, during bacterial infection, this innate immune response warns the body that a bacterial infection is present, causing the immune system to mount an antimicrobial attack. However, an overwhelming immune response to endotoxin can be pathological, leading to Systemic Inflammatory Response Syndrome and, possibly, shock, which can lead to multiple organ failure and, possibly, death.

Endotoxin has been suggested to be a causative agent of a large number of complications from surgery. However, while it has been clearly established that blood or plasma endotoxin can be detected during and after surgery, it is often found without a locus of bacterial infection (i.e., an endotoxin source) (Andersen et al., J. Thorac. Cardiovasc. Surg. 93(1): 115-119, 1987). It is now believed possible that this endotoxin may come from the gut (Martinez-Pellus et al., Intensive Care Med. 23(12):1251-1257, 1997). Gram-negative bacteria colonize the intestine. The ability of the mucosal barrier of the intestine to block translocation of endotoxin from inside the intestine to the blood supply may be compromised by gut ischemia or hypoperfusion. This occurs when blood circulation and oxygenation of the intestines is impaired (Oudemans-van Straaten et al., J. Cardiothorac. Vasc. Anesth. 10(2):187-194, 1996). Such ischemia can occur during cardiac failure (Niebauer et al., Lancet 353(9167):1838-1842, 1999) or during coronary artery bypass graft surgery (Martinez-Pellus et al., supra). Gut mucosal hypoperfusion has recently been called “the motor that drives multiple organ failure” (Chieveley-Williams et al., Int. Anesthesiol. Clin. 37(2):81-110, 1999). Still, despite the observation that endotoxin can be detected during and after surgery, it has been extremely difficult to establish a causal relationship between gut-derived endotoxin and complications following surgery.

Recently, a series of papers have described antibodies that cross-react with a wide variety of endotoxins from different bacteria. While most antibodies are species-specific (or even strain-specific) for endotoxin, the recently described antibodies react with the endotoxin core, which is common to endotoxins from a wide variety of gram-negative bacteria. This discovery of so-called endotoxin core antibodies (EndoCab) has enabled the research community to detect a relationship between levels of anti-endotoxin antibody and surgical outcome for coronary artery bypass graft surgery. In particular, when antibody levels to endotoxin are high prior to surgery, a patient may be able to neutralize or clear endotoxin during and after surgery. Prior to elective surgery, such as coronary artery bypass graft surgery, candidates with high EndoCab antibody titers, thus, are likely to have a greater chance of a surgical outcome lacking complications (e.g., death, requirement for intra-aortic balloon counter pulsation, requirement for chest re-opening other than for gross surgical bleeding, major organ failure, delay in ICU discharge of greater than 48 hours, or delay in hospital discharge of greater than 48 hours longer than anticipated) (Barclay, Prog. Clin. Biol. Res. 392:263-272, 1995; Bennett-Guerrero et al., J. American Medical Assoc. 277(8):646-650, 1997; Hamilton-Davies et al., Chest 112(5):1189-1196, 1997).

In a limited study, some evidence has been found that EndoCab antibody levels are also important for non-cardiac elective surgery (Mythen et al., Blood Coagul. Fibrinolysis 4(6):999-1005, 1993). Evidence is also building that endotoxin can complicate surgery for patients with acute pancreatitis (Windsor et al., Br. J. Surg. 80(8):1042-1048, 1993), inflammatory bowel disease (Gardiner et al., Gut 36(6):897-901, 1995), abdominal aortic aneurysm surgery (Soong et al., Crit. Care Med. 25(9):1472-1479, 1997), placement of a transjugular intrahepatic portosystemic stent shunt (Basili et al., Thromb. Haemot. 81(5):711-714, 1999), hepatic resection (Sato et al., Ther. Apher. 1(1):75-78, 1997), transplantation surgery (Miki et al., Arch. Surg. 132(2):136-141, 1997; Beebe et al., Transplant Proc. 27(1):593-594, 1995; Fryer et al., Arch. Surg. 131(1):77-84, 1996; Pirenne et al., Transplantation 61(12):1685-1694, 1996; Yokoyama et al., Transplant Proc. 21(5):3833-3841, 1989; Yokoyama et al., Hepatogastroenterology 42(3):205-208, 1995), burn wound revision (Ljunghusen et al., Inflammation 19(4):457-468, 1995), or burn wound escharectomy (Gao et al., Chung Hua Wai Ko Tsa Chih 34(7):443-446, 1996).

### SUMMARY OF THE INVENTION

The invention relates to the prevention and treatment of endotoxemia and related complications (e.g., sepsis syndrome, neurological complications, and renal complications (also see Grover et al., Ann. Thorac. Surg. 62(5 Suppl): S6-11, S31-2, 1996)) associated with surgical procedures, such as cardiac surgery, e.g., coronary artery bypass graft surgery and/or valve replacement surgery, or surgery with cardiopulmonary bypass. In these methods, an antiendotoxin compound is administered (e.g., intravenously) to a patient, before, during, and/or after surgery. The antiendotoxin compound can have the formula:

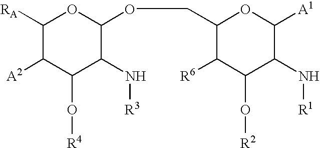

where R¹ is selected from the group consisting of:

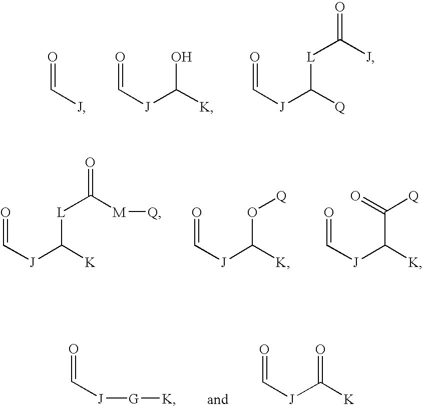

where each J, K, and Q, independently, is straight or branched C1 to C15 alkyl;

L is O, NH, or CH₂; M is O or NH; and G is NH, O, S, SO, or SO₂;

R² is straight or branched C5 to C15 alkyl;

R³ is selected from the group consisting of straight or branched C5 to C18 alkyl,

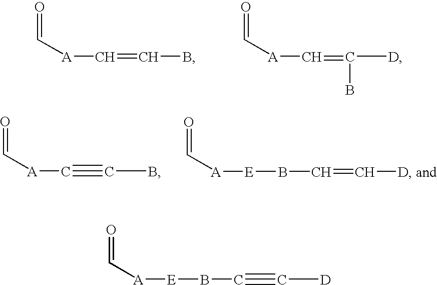

where E is NH, O, S, SO, or SO₂; each A, B, and D, independently, is straight or branched C1 to C15 alkyl;

R⁴ is selected from the group consisting of straight or branched C4 to C20 alkyl, and

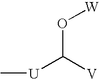

where each U and V, independently, is straight or branched C2 to C15 alkyl and W is hydrogen or straight or branched C1 to C5 alkyl;

R_{A} is R⁵ or R⁵—O—CH₂—, R⁵ being selected from the group consisting of hydrogen, J′, -J′-OH, -J′-O—K′, -J′-O—K′—OH, and -J′-O—PO(OH)₂, where each J′ and K′, independently, is straight or branched C1 to C5 alkyl;

R⁶ is selected from the group consisting of hydroxy, halogen, C1 to C5 alkoxy and C1 to C5 acyloxy;

A¹ and A², independently, are selected from the group consisting of

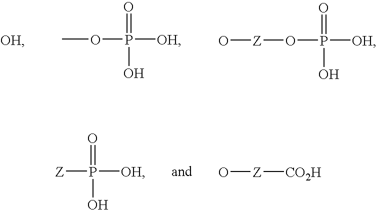

where Z is straight or branched C1 to C10 alkyl;

or pharmaceutically acceptable salts thereof.

A preferred, specific example of a compound that can be used in the invention has the following structure:

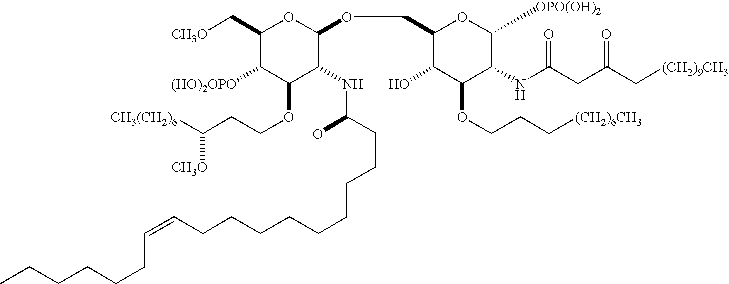

The antiendotoxin compound can be administered, for example, intravenously to the patient, in a dosage of about 0.002-10, 0.025-5, or 0.5-3 mg/hour, by bolus injection or infusion for 0.5-6 hours preoperatively and, after continuing through surgery, for up to 72 hours postoperatively. Alternatively, the antiendotoxin compound can be administered in a dosage of about 0.5, 3, or 7 mg/hour for about 4 hours, beginning about 1 hour before surgery.

Other features and advantages of the invention will be apparent from the following detailed description thereof.

### DETAILED DESCRIPTION

The invention provides methods of preventing and treating endotoxemia and related complications associated with surgery, such as cardiac surgery, e.g., coronary artery bypass graft surgery and/or valve replacement surgery. As is discussed above, endotoxin has been suggested to play a role in a large number of complications arising from surgical procedures. According to the present invention, an antiendotoxin compound, such as Compound 1287 (EE564; SGEA) or Compound B531, is administered to a patient before, during, and/or after surgery to prevent or treat the effects of endotoxemia that has occurred as a result of surgery.

The methods of the invention can be used in conjunction with any type of surgery or medical procedure that could lead to the occurrence of endotoxemia or related complications (e.g., organ dysfunction or sepsis syndrome). For example, the methods of the invention can be used in conjunction with cardiac surgery (e.g., cardiopulmonary bypass and/or valve replacement), transplantation (of, e.g., liver, heart, kidney, or bone marrow), cancer surgery (e.g., removal of a tumor), or any abdominal surgery. Additional examples of surgical procedures with which the methods of the invention can be used are surgery for treating acute pancreatitus or inflammatory bowel disease, placement of a transjugular intrahepatic portosystemic stent shunt, hepatic resection, burn wound revision, and burn wound escharectomy.

The methods of the invention can also be used in conjunction with non-surgical procedures in which the gastrointestinal tract is compromised. For example, the methods of the invention can be used in association with chemotherapy or radiation therapy with or without bone marrow transplant in the treatment of cancer.

Antiendotoxin compounds that can be used in the methods of the invention include, for example, Compound 1287 (EE564; SGEA) (U.S. Pat. No. 5,935,938; see structure, above) and Compound B531 (U.S. Pat. No. 5,530,113), as well as other compounds that are described in these patents and the following U.S. patents: U.S. Pat. No. 5,612,476, U.S. Pat. No. 5,756,718, U.S. Pat. No. 5,843,918, U.S. Pat. No. 5,750,664, and U.S. Pat. No. 5,681,824.

Antiendotoxin compounds can be administered according to the methods of the invention using routes (e.g., injection or infusion) and dosages that are determined to be appropriate by those of skill in this art. For example, the drug can be administered intravenously for 0.5-6 hours preoperatively, and administration can be continued through surgery and for up to 72 hours (e.g., 24 hours) postoperatively. The dose can be, for example, 0.002-10 mg/hour, e.g., 0.025-5 mg/hour or 0.5-3 mg/hour. Alternatively, the drug can be administered only preoperatively, operatively, or postoperatively, or in any combination thereof. In another example, the drug is administered at a dosage of about 0.25-5 mg/kg (e.g., 0.5, 1, 2.5, or 5 mg/kg), commencing before surgery (e.g., 0.5-2 hours before surgery), and continuing through surgery for a total time of administration of about 2.5-6 (e.g., 4-5) hours. As a specific example, the drug can be administered at a dosage of 0.5, 3, or 7 mg/hour for about 4 hours, beginning at about 1 hour before surgery and continuing into (and possibly beyond) the time of the surgery.

The drug is typically administered in a pharmaceutically acceptable formulation, e.g., dissolved in a physiological solution, which may include 5% glucose, or any other physiologically compatible infusion solutions.

All publications cited above are hereby incorporated by reference.

What is claimed is:

1. A method of preventing or treating endotoxemia in a cardiac surgical patient, said method comprising administering an antiendotoxin compound to said patient.

2. The method of claim 1, wherein said cardiac surgical patient has had, is having, or will be having coronary artery bypass graft surgery.

3. The method of claim 1, wherein said cardiac surgical patient has had, is having, or will be having valve replacement surgery.

4. The method of claim 1, wherein said cardiac surgical patient has had, is having, or will be having surgery with cardiopulmonary bypass.

5. The method of claim 1, wherein said antiendotoxin compound is of the formula: 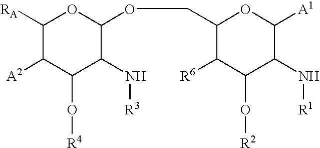
where R¹ is selected from the group consisting of 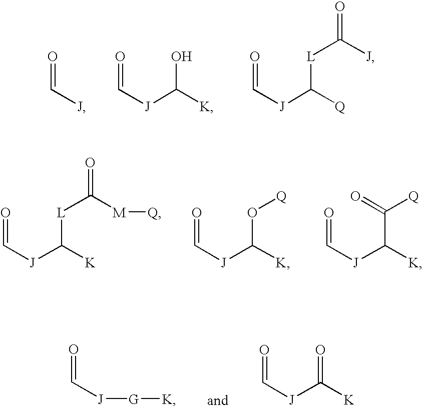
where each J, K, and Q, independently, is straight or branched C1 to C15 alkyl;
L is O, NH, or CH₂; M is O or NH; and G is NH, O, S, SO, or SO₂;
R² is straight or branched C5 to C15 alkyl;
R³ is selected from the group consisting of straight or branched C5 to C18 alkyl, 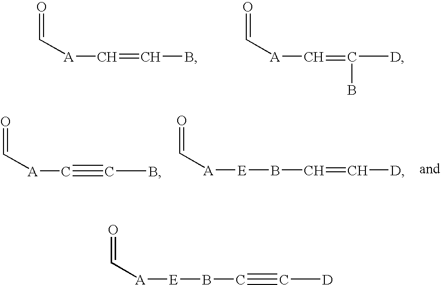
where E is NH, O, S, SO, or SO₂; each A, B, and D, independently, is straight or branched C1 to C15 alkyl;
R⁴ is selected from the group consisting of straight or branched C4 to C20 alkyl, and 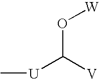
where each U and V, independently, is straight or branched C2 to C15 alkyl and W is hydrogen or straight or branched C1 to C5 alkyl;
R_{A} is R⁵ or R⁵—O—CH₂—, R⁵ being selected from the group consisting of hydrogen, J′, -J′-OH, -J′-O—K′, -J′-O—K′—OH, and -J′-O—PO(OH)₂, where each J′ and K′, independently, is straight or branched C1 to C5 alkyl;
R⁶ is selected from the group consisting of hydroxy, halogen, C1 to C5 alkoxy and C1 to C5 acyloxy;
A¹ and A², independently, are selected from the group consisting of 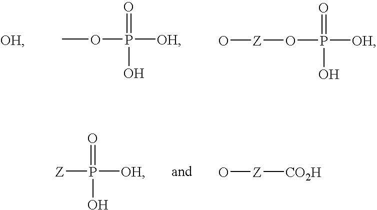
where Z is straight or branched C1 to C10 alkyl;
or pharmaceutically acceptable salts thereof.

6. The method of claim 5, wherein said antiendotoxin compound has the following structure:

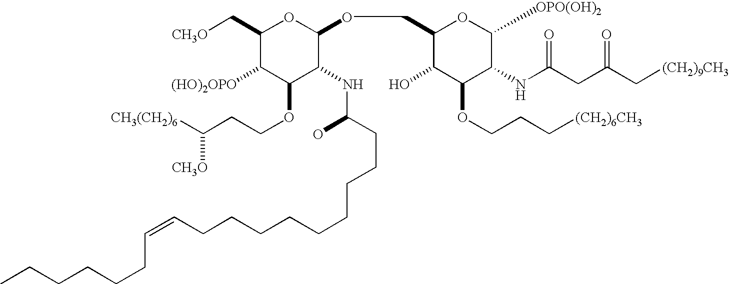

7. The method of claim 1, wherein said antiendotoxin compound is administered intravenously to said patient.

8. The method of claim 1, wherein said antiendotoxin compound is administered to said patient in a dosage of 0.002-10 mg/hour.

9. The method of claim 8, wherein said antiendotoxin compound is administered to said patient in a dosage of 0.025-8 mg/hour.

10. The method of claim 9, wherein said antiendotoxin compound is administered to said patient in a dosage of 0.5-3 mg/hour.

11. The method of claim 8, 9, or 10, wherein administration of said antiendotoxin to said patient is commenced 0.5-6 hours prior to surgery and continues for up to 72 hours postoperatively.

12. The method of claim 1, wherein said antiendotoxin compound is administered to said patient for about 4 hours at a dosage of about 0.5 mg/hour.

13. The method of claim 1, wherein said antiendotoxin compound is administered to said patient for about 4 hours at a dosage of about 3 mg/hour.

14. The method of claim 1, wherein said antiendotoxin compound is administered to said patient for about 4 hours at a dosage of about 7 mg/hour.

15. The method of claim 12, 13, or 14, wherein administration of said antiendotoxin compound to said patient is commenced about 1 hour prior to surgery.
